(19) United States
(12) Patent Application Publication
(10) Pub. No.: US 2023/0323306 A1
(43) Pub. Date: Oct. 12, 2023

(54) JAMMED GEL BIOINK WITH SELF-ASSEMBLING PEPTIDES

(71) Applicants: 3-D Matrix, Ltd., Cambridge, MA (US); CENTRE NATIONAL DE LA RECHERCHE SCIENTIFIQUE (CNRS), PARIS CEDEX 16 (FR); UNIVERSITE CLAUDE BERNARD LYON 1 (UCBL1), VILLEURBANNE (FR); L’ECOLE SUPERIEURE DE CHIMIE, PHYSIQUE, ELECTRONIQUE DE LYON (CPE Lyon), Villeurbanne Cedex (FR)

(72) Inventors: Christophe A. Marquette, Villeurbanne (FR); Marie Reveiller Buffier, Fontaines Saint Martin (FR); Marika G. Rioult, Cambridge, MA (US); Eun Seok Gil, Acton, MA (US)

(73) Assignees: 3-D Matrix, Ltd., Tokyo (JP); CENTRE NATIONAL DE LA RECHERCHE SCIENTIFIQUE (CNRS), 75794 PARIS CEDEX 16 (FR); UNIVERSITE CLAUDE BERNARD LYON 1 (UCBL1), VILLEURBANNE (FR); L’ECOLE SUPERIEURE DE CHIMIE, PHYSIQUE, ELECTRONIQUE DE LYON (CPE Lyon), Villeurbanne Cedex (FR)

(21) Appl. No.: 18/296,168

(22) Filed: Apr. 5, 2023

Related U.S. Application Data
(60) Provisional application No. 63/327,830, filed on Apr. 6, 2022.

(51) Int. Cl. C12N 5/077; B33Y 70/00

(52) U.S. Cl. CPC C12N 5/0656; C12N 2501/999; C12N 2513/00; B33Y 70/00

(57) ABSTRACT

A jammed gel bioink comprises a self-assembling peptide solution comprising a self-assembling peptide, and a cell solution comprising living cells and a cell culture medium. The self-assembling peptide solution is extruded to form an object, and then the cell solution is injected into the object. Useful methods of making the jammed gel bioink are also disclosed.

### PRIORITY

This application claims priority to U.S. provisional Application No. 63/327,830, filed Apr. 6, 2022, the entire contents of which are hereby incorporated by reference.

### SEQUENCE LISTING

The instant application contains an XML Sequence Listing which has been submitted electronically and is hereby incorporated by reference in its entirety. The Sequence Listing, created on Mar. 20, 2023, is named 3DM-21-02-3DP2-US-SL.xml and is 30,433 bytes in size.

### FIELD OF THE INVENTION

This invention relates to bioinks suitable for 3D printing, and more particularly to improved jammed gel bioinks containing self-assembling peptides.

### BACKGROUND OF THE INVENTION

3-D printing of tissues involves the assembly of cells into a controlled volume, where the bioink mimics in vivo characteristics of tissues. There are competing design constraints which such bioinks. For example, a particular shape is desired, and so the material used to make that shape must be extrudable but also ultimately capable of holding that shape. But adding the requirement of living cells means the material used and the way the material is extruded must also be non-toxic to the living cells. Various efforts are known to make bioinks. These include seeding cells on printed structures, mixing cells with a printing medium and then printed into desired structures, and printing cell clusters (spheroids) and stringing them together to form functional tissues. As this technology is fairly new, different printers and printing techniques have been developed to print the cells and the matrix. The commonly used methods of bioprinting are extrusion, laser, inkjet, and tissue fragment printing, all with a goal of positioning of the living cells and/or the biomaterials, and to create functional tissue analogs.

Many different materials have been used as bioinks, including, for example, natural materials such as alginate, gelatin, collagen, silk, gellan gum, hyaluronic acid, dextran, and cellulose; synthetic materials like polycaprolactone, pluronic acid and polyethylene glycol; and commercial materials like Derma Matrix®, Novogel®, and CELLINK®. Jammed bioinks should be printable, with easy and cell friendly cross-linking after printing/extrusion so as to retain stiffness quickly after extrusion, should hold its shape so as to mimic the desired shape with precision, should be biocompatible to not just allow cells to live but also allow for cell growth and not cause an immune response, and should biodegradable. Further, such jammed bioinks should preferably mimic the native tissue environment for cells to attach and/or grow within, and such jammed bioinks should be customizable so as to be modifiable for different applications (such as soft, medium or hard tissues). In spite of the numerous efforts in the advancement of the bioprinting technology, the development of a satisfactory jammed bioink which meets all the requirements to create a biomaterial serving as a suitable functional tissue analog has been limited. It would be desirable to provide a jammed bioink which goes further towards satisfying these competing design constraints.

### SUMMARY OF THE INVENTION

In accordance with a first aspect, there is provided a jammed gel bioink comprising a self-assembling peptide solution comprising a self-assembling peptide, and a cell solution comprising living cells and a cell culture medium. The self-assembling peptide solution is extruded to form an object, and then the cell solution is injected into the object. Useful methods of making the jammed gel bioink are also disclosed.

From the foregoing disclosure and the following more detailed description of various embodiments it will be apparent to those skilled in the art that the present invention provides a significant advance in the technology of jammed gel bioinks. Particularly significant in this regard is the potential the invention affords for providing a high-quality jammed gel bioink which is easily extrudable, of a customizable shaped object and which is non-toxic to living cells. Additional features and advantages of various embodiments will be better understood in view of the detailed description provided below.

### DETAILED DESCRIPTION OF CERTAIN EMBODIMENTS

It will be apparent to those skilled in the art, that is, to those who have knowledge or experience in this area of technology, that many uses and design variations are possible for the jammed gel bioink disclosed here. The following detailed discussion of various alternate features and embodiments will illustrate the general principles of the invention with reference to 3-D printing of jammed gel bioinks, and more particularly to medical and research applications of such jammed bioinks, formed as jammable gels, where the cell solution is at least partially surrounded by the self-assembling peptide solution. Other embodiments suitable for other applications will be apparent to those skilled in the art given the benefit of this disclosure.

In accordance with a first aspect, there is provided a jammed gel bioink, or more simply a jammed bioink, is typically formed when a self-assembling peptide solution is extruded first to form a shaped object, and then a cell solution comprising living cells is injected into the object such that the cell solution is at least partially and or even entirely surrounded by the self-assembling peptide solution. The self-assembling peptide solution comprises one of many self-assembling peptides, for example, RADA16, which a peptide of 16 amino acids in length with protective groups on both ends (AcN-RADARADARADARADA-CNH₂). An example of a suitable, commercially available, self-assembling peptide solution is PuraStat®, from 3-D Matrix Medical Technology, Inc., which is an aqueous solution of about 2.5% RADA16. RADA16 has alternating hydrophobic (alanine) and hydrophilic (arginine and aspartic acid) groups which allow for organization into stable β-sheets. The resulting porous hydrogels contain high water content (on the order of 99%) and resemble the extracellular matrix of native body tissue. Optionally a sucrose solution may be incorporated into the self-assembling peptide solution. Other self-assembling peptides, including shorter RADA derivatives (RADAR, RADA12, for example), peptidomimetics (such as D-forms of the amino acids, for example), peptide amphiphiles, other concentrations, and other self-assembling peptide solutions suitable for use in a jammed gel bioink will be readily apparent to those skilled in the art given the benefit of this disclosure.

In some embodiments, the SAPs comprise a sequence of amino acid residues conforming to one or more of Formulas I-IV:

((Xaaneu-Xaa+)x(Xaaneu-Xaa−)y)n (I)

((Xaaneu-Xaa−)x(Xaaneu-Xaa+)y)n (II)

((Xaa+-Xaaneu)x(Xaa−-Xaaneu)y)n (III)

((Xaa−-Xaaneu)x(Xaa+-Xaaneu)y)n (IV)

Xaaneu represents an amino acid residue having a neutral charge; Xaa+ represents an amino acid residue having a positive charge; Xaa− represents an amino acid residue having a negative charge; x and y are integers having a value of 1, 2, 3, or 4, independently; and n is an integer having a value of 1-5. In some embodiments, the SAPs further comprise an amino acid sequence that interacts with the extracellular matrix, wherein the amino acid sequence anchors the SAPs to the extracellular matrix.

In some embodiments, the specific peptides for use in the method of the present invention can be chosen from one or more peptides listed in the Table 1 below.

TABLE 1

| Self-assembling Peptide | SEQ ID NO: |
|---|---|
| RADARADARADARADA | SEQ ID NO: 1 |
| KLDKLDKLDKLD | SEQ ID NO: 2 |
| IEIKIEIKIEIKI | SEQ ID NO: 3 |
| QLELQLELQLEL | SEQ ID NO: 4 |
| ADARADARADARADAR | SEQ ID NO: 5 |
| RAEARAEARAEARAEA | SEQ ID NO: 6 |
| RVDVRVDVRVDVRVDV | SEQ ID NO: 7 |
| RLDLRLDLRLDLRLDL | SEQ ID NO: 8 |
| RIDIRIDIRIDIRIDI | SEQ ID NO: 9 |
| RFDFRFDFRFDFRFDF | SEQ ID NO: 10 |
| AEARAEARAEARAEAR | SEQ ID NO: 11 |
| KADAKADAKADAKADA | SEQ ID NO: 12 |
| KIDIKIDIKIDIKIDI | SEQ ID NO: 13 |
| KIEIKIEIKIEIKIEI | SEQ ID NO: 14 |
| IDIKIDIKIDIKI | SEQ ID NO: 15 |
| IEIRIEIRIEIRI | SEQ ID NO: 16 |
| LELKLELKLELKL | SEQ ID NO: 17 |
| FEFKFEFKFEFKF | SEQ ID NO: 18 |
| KLDLKLDLKLDL | SEQ ID NO: 19 |
| KLELKLELKLEL | SEQ ID NO: 20 |
| FEFRFEFRFEFRF | SEQ ID NO: 21 |
| YEYKYEYKYEYKY | SEQ ID NO: 22 |
| WEWKWEWKWEWKW | SEQ ID NO: 23 |

In some embodiments, the amino acid residues in the SAPs can be naturally occurring or non-naturally occurring amino acid residues. Naturally occurring amino acids can include amino acid residues encoded by the standard genetic code as well as non-standard amino acids (e.g., amino acids having the D-configuration instead of the L-configuration), as well as those amino acids that can be formed by modifications of standard amino acids (e.g., pyrolysine or selenocysteine). Suitable non-naturally occurring amino acids include, but are not limited to, D-alloisoleucine(2R,3S)-2-amino-3-methylpentanoic acid, L-cyclopentyl glycine (S)-2-amino-2-cyclopentyl acetic acid.

In other embodiments, another class of materials that can self-assemble are peptidomimetics. Peptidomimetics, as used herein, refers to molecules which mimic peptide structure. Peptidomimetics have general features analogous to their parent structures, polypeptides, such as amphiphilicity. Examples of such peptidomimetic materials are described in Moore et al., Chem. Rev. 101(12), 3893-4012 (2001). The peptidomimetic materials can be classified into four categories: α-peptides, β-peptides, γ-peptides, and δ-peptides. Copolymers of these peptides can also be used. Examples of α-peptide peptidomimetics include, but are not limited to, N,N′-linked oligoureas, oligopyrrolinones, oxazolidin-2-ones, azatides and azapeptides. Examples of β-peptides include, but are not limited to, β-peptide foldamers, α-aminoxy acids, sulfur-containing β-peptide analogues, and hydrazino peptides. Examples of γ-peptides include, but are not limited to, γ-peptide foldamers, oligoureas, oligocarbamates, and phosphodiesters. Examples of δ-peptides include, but are not limited to, alkene-based δ-amino acids and carbopeptoids, such as pyranose-based carbopeptoids and furanose-based carbopeptoids.

In certain embodiments, the SAP is AC5®, AC5-V® or AC5-G™ made by Arch Therapeutics, Inc. (see www.archtherapeutics.com).

The cell solution may comprise any one of several living cells or combinations of living cells. Examples are numerous and comprise stomal vascular fraction (SVF) cells, stem cells, tumor cells, hepatocyte progenitor cells, rat pheochromocytoma cells (PC12), hippocampal neurons, endothelial cells, neuronal cells, fibroblasts, keratinocytes, and transformed cells including, for example, MG-62, SH-SY5Y, HEK293, NIH3T3). Other cells suitable for use in the cell solution of the jammed gel bioinks disclosed herein will be readily apparent to those skilled in the art given the benefit of this disclosure.

The cell solution would typically contain a cell culture medium to provide nutrition to the cells and maintain a healthy environment. Suitable components of a cell culture medium can comprise, for example, Dulbecco's Modified Eagle Medium (DMEM), which is a widely used basal medium for supporting the growth of many different mammalian cells, fetal bovine serum, Minimal Essential Medium (MEM) nonessential amino acids, L-glutamine solution, antibiotics such as penicillin and streptomycin, and a trypsin-EDTA (Ethylenediaminetetraacetic acid) solution. Other cell culture mediums components suitable for use in the cell solution of the jammed gel bioinks disclosed herein will be readily apparent to those skilled in the art given the benefit of this disclosure.

In accordance with one embodiment, the jammed gel bioink can be formed by initially extruding the self-assembling peptide solution, and then adding the cell solution. Alternatively, the self-assembling peptide solution and the cell solution may be coextruded together. In this latter case, the self-assembling peptide solution and the cell solution are not mixed together before or during extrusion, such that they remain essentially grouped together but unmixed. For example, during co-extrusion, the cell solution can be positioned in the center of the nozzle and the self-assembling peptide solution can surround the cell solution and during extrusion this relationship between the two solutions stays the same. Objects co-extruded this way can have an interior of a cell solution and an exterior of the self-assembling peptide solution. Extrusion or co-extrusion can occur continuously. Continuously is understood here to mean that the addition of the self-assembling peptide solution (or a co-extruded combination of both) to the extruder forces or urges a jammed gel bioink to be extruded from the extruder. This of course assumes that a control valve on a nozzle of the extruder is open to allow the extruded solutions to flow from the chamber and out the nozzle. Regardless of whether the cell solution is co-extruded with the self-assembling peptide solution or injected after formation of the object, much of the cell solution can wind up surrounding the self-assembling peptide solution. Surrounding is understood here to mean the cell solution is mostly surrounded or entirely surrounded by the self-assembling peptide solution (either alone or in combination with a tray or floor).

The extruder may have a chamber which receives both the self-assembling peptide solution and a consolidation solution (discussed below) where mixing occurs, or where co-extrusion occurs by aligning each solution prior to exiting the nozzle. Generally, as the pH of the self-assembling peptide solution rises, as by mixing with the consolidation solution to form the object created by the jammed gel bioink, cross linking occurs. It is preferable to extrude quickly such that most of the cross linking occurs after the self-assembling peptide solution has been extruded and formed a shaped object. In normal operation the self-assembling peptide solution is continuously introduced together and mixed, and the act of introducing more of the self-assembling peptide solution together forces the bioink to be extruded through a nozzle into an object having a desired shape. Suitable examples of a extruder can comprise, for example, the T333 classical FDM (“fused deposition modeling”) 3-D printer made by the French corporation Tobeca, provided with a chamber to optionally receive more than one solution, and an adjacent 1 mm nozzle such that introduction of more of either the self-assembling peptide solution, the cell solution, or both immediately forces the bioink through the nozzle. The volume of mixture can be controlled, along with the location, and can be controlled concurrently. Other extruders and 3-D printers suitable for mixing and extruding bioinks will be readily apparent to those skilled in the art given the benefit of this disclosure. Optionally the bioink may be immersed in an immersion solution after extrusion to provide additional nutrients to help keep the cells in the bioink alive.

Optionally when printing the self-assembling peptide solution alone, the consolidation solution composed of at least a basic solution such as NaOH 1N can be used. A ratio of 0.5-1.5/100 (consolidation solution/self-assembling peptide solution, such as NaOH/RADA16) can be used in the extruder, more preferably 1/100 (NaOH/RADA16), and preferably mixed together. The consolidation solution can be loaded, under sterile conditions, in a 10 mL syringe just before being connected to the mixing extruder. Advantageously, the bioink preferably can be free of any additional thickening agent (such as, for example, methylcellulose), as the cross-linking of the self-assembling peptide solution creates an object with sufficient rigidity/viscosity for many applications. Further, the self-assembling peptide solution may be kept chilled or may be at room temperature (that is, at least above 5° C., above 10° C., or above 15° C.) prior to mixing with the cell solution, and/or after mixing with the cell solution, but before the step of extruding the bioink. The cell solution can be kept at the temperature the cells normally live in, which for cells that live in a human is around 35-40° C., and more specifically near 37° C., for example. The jammed gel bioink can be a mixture of the self-assembling peptide solution and the basic solution which cures to form an object having a desired or pre-programmed shape. The object cures spontaneously after extrusion, without additional heating or other process steps by an operator. Curing of the object is understood here to mean extensive cross linking occurs in the self-assembling peptide solution, either with or without addition of the consolidation solution, and either before or after the cell solution is added to the object.

For jammed gel bioinks, the cell solution may be injected or introduced into the self-assembling peptide solution, without mixing, and optionally before or after curing of the self-assembling peptide solution occurs. When the cell solution is largely surrounded by the self-assembling peptide solution, the cell solution can be protected from air and environmental contaminants. This can be desirable for some types of cells. Advantageously the cells in the cell solution can be kept alive for at least 5 days, at least 7 days, or at least 8 days after the steps of extruding and injecting. Preferably during both the steps of extruding and injecting a shear stress on the bioink is kept below a viability limit for the living cells, such as, for example, 4000 PA. The jammed gel bioink may also be at least partially immersed in an immersion solution after extrusion to provide additional nutrients to help keep the cells in the jammed gel bioink alive. The immersion solution can comprise, for example, a basal medium for supporting the growth of many different types of cells, especially mammalian cells, such as Dulbecco's Modified Eagle Medium (DMEM). Other compositions and combinations of chemicals suitable for use as the immersion solution will be readily apparent to those skilled in the art given the benefit of this disclosure.

EXAMPLE 1. One example of a jammed bioink is as follows. RADA16 was used as the self-assembling peptide and deposited in a 6-well Transwell® insert from Corning (USA) using the manufacturer's syringe and disposable tip. The RADA16 loaded inserts were then placed in a 6-well plate and in turn, the plate was positioned inside a BioAssembly Bot bioprinter from Advanced Solution Life Science (USA). The 6-axis robotic arm-based bioprinter was then used to print in jammed gel the cell solution within the RADA16. Prior to printing the self-assembling peptide solution, green fluorescent protein (GFP)-expressing fibroblasts (NIH3T3/GFP, AKR-214 from Cell Biolabs Inc. (US) were expanded and suspended in DMEM (high glucose) from Gibco (France), supplemented with 10% (v/v) fetal bovine serum (FBS), from Gibco (France), 0.1 mM MEM non-essential amino acids (NEAA) from Invitrogen (France), 2 mM L-glutamine from Gibco (France) and 1% (w/v) penicillin/streptomycin (10,000 U/mL) from Gibco (France). Before printing in jammed bioink, the cells in the cell solution were trypsinized (0.25% (v/v) trypsin-EDTA from ThermoFisher, (France)), and counted. Cells in the cell solution were about 6×10⁶ cells/mL. The cell solution was loaded into a syringe, which in turn was loaded into a 6-axis robotic bioprinter and extruded/printed into the self-assembling peptide solution using a nozzle 450 μm (length 6 mm). After printing, the wells of the 6-well plate were filled with an immersion solution comprising 5 mL of DMEM (high glucose) from Gibco (France) supplemented with 10% (v/v) fetal bovine serum (FBS) from Gibco (France), 0.1 mM MEM non-essential amino acids (NEAA) from Invitrogen (France), 2 mM L-glutamine from Gibco (France) and 1% (w/v) penicillin/streptomycin (10,000 U/mL) from Gibco (France), and placed at 37° C. in a 5% CO2 incubator to help keep the cells alive. Cell fluorescence analysis from Day 2 to Day 8 demonstrated the cells were viable after 8 days and that the applied path of the cell solution was also preserved during these 8 days. While most of the cells are located within the printed paths, a non-negligible number of cells “leaked” from the path and are also found in the surrounding gel. This phenomenon was found to be directly linked to the printing in jammed bioink protocol in which the printing nozzle delivery the cell solution travels down inside the self-assembling peptide solution formed as an object, and then prints while generating crevasses. Upon withdrawal of the printing nozzle, a leaking path of cells is formed. Overall, the cells in the jammed bioink were growing and proliferating well within the 3D printed matrix of the self-assembling peptide solution.

From the foregoing disclosure and detailed description of certain embodiments, it will be apparent that various modifications, additions and other alternative embodiments are possible without departing from the true scope of the invention. The embodiments discussed were chosen and described to provide the best illustration of the principles of the invention and its practical application to thereby enable one of ordinary skill in the art to use the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A jammed gel bioink comprising, in combination:
a self-assembling peptide solution comprising a self-assembling peptide; and
a cell solution comprising living cells and a cell culture medium;
wherein the self-assembling peptide solution is extruded to form an object, and then the cell solution is injected into the object.

2. The jammed gel bioink of claim 1, wherein the self-assembling peptide solution comprises a self-assembling peptide chosen from Table 1.

3. The jammed gel bioink of claim 2, wherein the cell culture medium comprises DMEM.

4. The jammed gel bioink of claim 1, further comprising an immersion solution in which the object is at least partially immersed after extrusion.

5. The jammed gel bioink of claim 1, further comprising a consolidation solution added to the self-assembling peptide solution, wherein the consolidation solution is extruded with the self-assembling peptide solution.

6. The jammed gel bioink of claim 5 wherein a ratio of the consolidation solution to the self-assembling peptide solution is 0.5-1.5/100 by volume.

7. The jammed gel bioink of claim 5, wherein the consolidation solution is a basic solution.

8. The jammed gel bioink of claim 7 wherein the basic solution is an aqueous NaOH solution.

9. The jammed gel bioink of claim 1, wherein the bioink is free of a thickening agent.

10. The jammed gel bioink of claim 1, wherein the self-assembling peptide solution is formed as a cured object, and surrounds the cell solution.

11. The jammed gel bioink of claim 1, wherein the self-assembling peptide solution comprises RADA16 (SEQ ID NO:1).

12. A method of making a jammed gel bioink comprising, in combination, the steps of:
extruding a self-assembling peptide solution comprising a self-assembling peptide, to form an object;
injecting a cell solution comprising living cells and a cell culture medium into the object; and
curing the object.

13. The method of claim 12, wherein during injection of the cell solution, a shear stress on the cell solution is kept below a viability limit for the living cells.

14. The method of claim 13, wherein the viability limit is 4000 PA.

15. The method of claim 12, wherein the cell solution is injected into the object before the object cures.

16. The method of claim 12, further comprising the step of mixing a consolidation solution with the self-assembling peptide solution prior to extrusion.

17. The method of claim 12 wherein the self-assembling peptide solution is refrigerated prior to extruding, and the cell solution is kept at 35-40° C. prior to injecting into the self-assembling peptide solution.

18. A method of making a jammed gel bioink comprising, in combination, the steps of:
extruding a self-assembling peptide solution comprising a self-assembling peptide, together with a cell solution comprising living cells and a cell culture medium to form an object;
wherein the self-assembling peptide solution and the cell solution are not mixed together before or during extrusion; and
curing the object.


SEQUENCE LISTING (23 sequences)

SEQ ID NO: 1 - protein, 16 aa, Synthetic construct
  REGION 1..16: note = Description of Artificial Sequence: Synthetic Peptide
RADARADARADARADA

SEQ ID NO: 2 - protein, 12 aa, Synthetic construct
  REGION 1..12: note = Description of Artificial Sequence: Synthetic Peptide
KLDKLDKLDKLD

SEQ ID NO: 3 - protein, 13 aa, Synthetic construct
  REGION 1..13: note = Description of Artificial Sequence: Synthetic Peptide
IEIKIEIKIEIKI

SEQ ID NO: 4 - protein, 12 aa, Synthetic construct
  REGION 1..12: note = Description of Artificial Sequence: Synthetic Peptide
QLELQLELQLEL

SEQ ID NO: 5 - protein, 16 aa, Synthetic construct
  REGION 1..16: note = Description of Artificial Sequence: Synthetic Peptide
ADARADARADARADAR

SEQ ID NO: 6 - protein, 16 aa, Synthetic construct
  REGION 1..16: note = Description of Artificial Sequence: Synthetic Peptide
RAEARAEARAEARAEA

SEQ ID NO: 7 - protein, 16 aa, Synthetic construct
  REGION 1..16: note = Description of Artificial Sequence: Synthetic Peptide
RVDVRVDVRVDVRVDV

SEQ ID NO: 8 - protein, 16 aa, Synthetic construct
  REGION 1..16: note = Description of Artificial Sequence: Synthetic Peptide
RLDLRLDLRLDLRLDL

SEQ ID NO: 9 - protein, 16 aa, Synthetic construct
  REGION 1..16: note = Description of Artificial Sequence: Synthetic Peptide
RIDIRIDIRIDIRIDI

SEQ ID NO: 10 - protein, 16 aa, Synthetic construct
  REGION 1..16: note = Description of Artificial Sequence: Synthetic Peptide
RFDFRFDFRFDFRFDF

SEQ ID NO: 11 - protein, 16 aa, Synthetic construct
  REGION 1..16: note = Description of Artificial Sequence: Synthetic Peptide
AEARAEARAEARAEAR

SEQ ID NO: 12 - protein, 16 aa, Synthetic construct
  REGION 1..16: note = Description of Artificial Sequence: Synthetic Peptide
KADAKADAKADAKADA

SEQ ID NO: 13 - protein, 16 aa, Synthetic construct
  REGION 1..16: note = Description of Artificial Sequence: Synthetic Peptide
KIDIKIDIKIDIKIDI

SEQ ID NO: 14 - protein, 16 aa, Synthetic construct
  REGION 1..16: note = Description of Artificial Sequence: Synthetic Peptide
KIEIKIEIKIEIKIEI

SEQ ID NO: 15 - protein, 13 aa, Synthetic construct
  REGION 1..13: note = Description of Artificial Sequence: Synthetic Peptide
IDIKIDIKIDIKI

SEQ ID NO: 16 - protein, 13 aa, Synthetic construct
  REGION 1..13: note = Description of Artificial Sequence: Synthetic Peptide
IEIRIEIRIEIRI

SEQ ID NO: 17 - protein, 13 aa, Synthetic construct
  REGION 1..13: note = Description of Artificial Sequence: Synthetic Peptide
LELKLELKLELKL

SEQ ID NO: 18 - protein, 13 aa, Synthetic construct
  REGION 1..13: note = Description of Artificial Sequence: Synthetic Peptide
FEFKFEFKFEFKF

SEQ ID NO: 19 - protein, 12 aa, Synthetic construct
  REGION 1..12: note = Description of Artificial Sequence: Synthetic Peptide
KLDLKLDLKLDL

SEQ ID NO: 20 - protein, 12 aa, Synthetic construct
  REGION 1..12: note = Description of Artificial Sequence: Synthetic Peptide
KLELKLELKLEL

SEQ ID NO: 21 - protein, 13 aa, Synthetic construct
  REGION 1..13: note = Description of Artificial Sequence: Synthetic Peptide
FEFRFEFRFEFRF

SEQ ID NO: 22 - protein, 13 aa, Synthetic construct
  REGION 1..13: note = Description of Artificial Sequence: Synthetic Peptide
YEYKYEYKYEYKY

SEQ ID NO: 23 - protein, 13 aa, Synthetic construct
  REGION 1..13: note = Description of Artificial Sequence: Synthetic Peptide
WEWKWEWKWEWKW